(19) United States
(12) Patent Application Publication
(10) Pub. No.: US 2009/0098619 A1
(43) Pub. Date: Apr. 16, 2009

(54) Method of producing liquid and powered mushroom beta-glucan

(76) Inventor: Shiu Nan Chen, San Marino, CA (US)

Correspondence Address: DAVID AND RAYMOND PATENT FIRM, 108 N. YNEZ AVE., SUITE 128, MONTEREY PARK, CA, 91754, US

(21) Appl. No.: 12/005,508

(22) Filed: Dec. 26, 2007

Related U.S. Application Data
(60) Provisional application No. 60/999,295, filed on Oct. 16, 2007.

(51) Int. Cl. C12P 19/04

(52) U.S. Cl. 435/101

(57) ABSTRACT

A method of producing liquid and powdered mushroom Beta-Glucan provides a uniform culture medium for the mushroom growing well and yielding high beta-glucan production rate. Adapting animal cell culture techniques, the growth of mushroom mycelium is speeded up and the production time is minimized. The use of serum bottles with magnetic stirring units reduces the size of mycelium to promote even distribution in result of better growth. The use of transparent, thermal resistant, and non-toxic polycarbonate (PC) bioreactors with orbital shakers, the fermentation process is better controlled and the production is enhanced while being production cost effective.

### CROSS REFERENCE OF RELATED APPLICATION

This is a non-provisional application of a provisional application having an application No. 60/999,295 and a filing date of Oct. 16, 2007.

### BACKGROUND OF THE PRESENT INVENTION

1. Field of Invention

The present invention relates to mushroom cultivation, and more particularly to a method of producing liquid and powered mushroom beta-glucan. A uniform liquid culture medium, along with the usage of serum bottles with magnetic stirring bars and non-toxic polycarbonate (PC) bioreactor is arranged to allow the mushrooms growing well and yielding high beta-glucan production rate, and thus reduce setup cost for cultivating liquid beta-glucan from mushrooms.

2. Description of Related Arts

Growing mushrooms with conventional fruiting bodies takes up a great deal of effort and time, usually around two to three months from cultivation to collection, and it may take even longer if the fruiting bodies are from outer fields. Thus, it is important to take advantages of liquid fermentation and the method of antibiotic fermentation when cultivating mushroom mycelium and other metabolites under the technique of submerged cultivation.

When the submerged cultivation method is used to produce mycelium and other metabolites, the factors such as composition of culture medium, pH value, temperature, aeration and mixing are important. The distinctive feature of the submerged cultivation method is that there is no sporulation period in the fermentation process and the mycelium is produced in the form of pellet. Therefore, the transportation of oxygen and nutrition is even more complicated than that in conventional liquid cultivation of lower fungi and yeast.

Generally speaking, the mycelium of mushroom grows slower in liquid fermentation, and longer fermentation period induces a higher possibility of contamination. Thus, higher inoculation rate (between 10% and 30%) is usually used in a large bioreactor to increase growing rate of the mycelium. However, some problems could be generated in the large bioreactor, such as insufficient mixing, and temperature, pH value and nutrition gradients. In addition, to minimize the problems previously mentioned, additional cultivation equipment is usually required.

During fermentation, mycelium bodies usually intertwine with each other to form mycelium pellets. The larger the mycelium pellet is, the more difficult the oxygen and nutrition can be transported to the center of the mycelium pellet, which creates a tough environment for the mushroom to grow and metabolize. Thus, during fermentation, it is critical to control the sheer force, mixing rate and the level of dissolved oxygen to minimize the size of mycelium pellets.

A new cultivating scheme of the present invention makes use of simple culture medium and low cost equipment to provide an optimum environment for mushroom growth. Also, the method not only successfully produces metabolites, such as mycelium and beta-glucan, but also overcomes the problems of dissolved oxygen and oversized mycelium pellets in submerged liquid culture.

### SUMMARY OF THE PRESENT INVENTION

A main object of the present invention is to provide a method to produce beta-glucan and other metabolites from all types of mushrooms via liquid culture, wherein a uniform liquid culture medium, along with the usage of serum bottles with magnetic stirring bars and non-toxic polycarbonate (PC) bioreactor is arranged to allow the mushrooms growing well and yielding high beta-glucan production rate, and thus reduce setup cost for cultivating liquid beta-glucan from mushrooms.

Another object of the present invention is to provide a method to produce beta-glucan and other metabolites from all types of mushrooms via liquid culture, wherein a serum bottle with a one-direction ventilating silicate stop and a magnetic Teflon spinner are used such that shear force created by the spinner makes the mycelium to densely grow and reproduce at a higher rate.

Another object of the present invention is to provide a method to produce beta-glucan and other metabolites from all types of mushrooms via liquid culture, wherein a polycarbonate (PC) bioreactor is used as a replacement of the expensive stainless bioreactor because polycarbonate material is light, non-toxic, thermal resistant and durable. Thus, during fermentation, the transparent PC bioreactor allows a user to clearly observe the fermentation process, such that the production process can be better controlled and screened.

In addition, it is worth to mention that as of today, most submerged liquid cultures for mushroom beta-glucan and other metabolites are produced in a sequence of bioreactors after initial mycelium production. Such production scheme involves massive investment in facility and the occurrence of the contamination during the process may incur great loss.

In mushroom liquid fermentation, carbon, nitrogen source and growth factor in the culturing medium are the key factors to the rate of beta-glucan production. Most production methods use cheaper materials or add some special compounds to increase the production rate, but this would result in more purification steps and creates additional waste. Therefore, it is another object of the present invention to adopts food-grade materials and follow simple recipe so that the culturing medium contains nothing more than glucose and yeast extracts. Experiments have shown that the present invention is suitable for all types of mushroom growth and the production rate of beta-glucan is good. After fermentation, the mushrooms only need minimum purification procedure for direct consumption, and with further processing, the mushrooms can become different products for medical, healthy or cosmetic use.

Agar plates are commonly used in subculturing methods. During fermentation, it is important to speed up the production of usable inoculums to further scale-up inoculation. It is another object of the present invention to provide a method of mushroom cultivation which uses the 80 T, the T-shaped tissue culture vessel for animal cell culture. The present invention has a faster and higher duplicating or reproducing rate, and if there is contamination, it can be detected at the early stage. Different from other liquid fermentation methods which shake the flasks, the present invention uses serum bottle with an air-permeable silica stop and a Teflon magnetic spinner for mixing. The present invention prompts extra-cellular beta-glucan production, reduces the formation of mycelium block and thus increases the amount of dissolved oxygen and material transportation rate.

Most conventional submerging culture methods use a stainless steel bioreactor with connection tubing to an industrial size boiler, and all of which contribute to a significant cost. In the present invention, culture medium is contained in a transparent and thermal resistive polycarbonate (PC) bioreactor and covered with the air-permeable silica stop and thus the bioreactor can be sterilized in a miniature autoclave. Such a bioreactor holds up to 16 L of fermentation volume, which is larger than conventional tabletop bioreactor. Furthermore, the PC bioreactors are lighter and cheaper than conventional bioreactors. After cooling down from the sterilization process, mushroom stains from the serum bottle are inoculated to the PC bioreactor placed on a custom-made rotating shaker. The rotating shaker can hold up to 16 bioreactors to increase the yields and more than one kinds of mycelium can be cultivated at the same time. The method of the present invention is extremely flexible and can be used for either research or manufacturing purpose. The transparent bioreactor allows the user to monitor the entire fermentation process. The shaking rate, amount of dissolved oxygen and growth conditions can all be adjusted accordingly. Experimental productions with mushroom-like Ganoderma lucidum, Coriolus Versicolor and Lentinula edodes have good yields and beta-glucan production is also shown to have good production rate.

The present invention is suitable for the production of beta-glucan and other metabolites from all types of mushroom via the liquid culture method. The spinning speed of the magnetic spinner and the liquid volume can be adjusted to control the property of dissolved oxygen. The producing method of the present invention is applicable to a wide range of cultivation, such as obligated aerobic, aerobic and anaerobic microbial fermentation, and for both animal and flora cell cultivation. The PC bioreactor 30 also allows liquid cultivation of algae and Rhodobacter Capsulatus under special light sources.

In order to accomplish the above objects and unexpected results, the present invention provides a method of producing mushroom beta-glucan, comprising the steps of:

(a) growing mycelium from mushroom strains;

(b) making a culturing liquid from the mycelium;

(c) disposing the culturing liquid in one or more bottles for first time fermentation under a first predetermined condition;

(d) after the culturing liquid is fermented, mixing and stirring the culturing liquid with a new culturing medium in each of the bottles at a predetermined spinning speed to control an amount of oxygen being dissolved in the culturing liquid and to create shear force to make the mycelium to densely grow and reproduce at a higher growth rate;

(e) second time fermenting the culturing liquid after the mycelium and beta-glucan reach a highest concentration levels thereof, wherein the mycelium is shredded to reduce a size thereof after having fermented twice;

(f) implanting the mycelium into one or more bioreactors which contains an extract liquid culturing medium; and

(g) fermenting the mycelium in said bioreactor under a predetermined condition, wherein the fermentation is stopped when a stationary phase is reached to obtain the highest concentration of mycelium cell and beta-glucan.

These and other objectives, features, and advantages of the present invention will become apparent from the following detailed description, the accompanying drawings, and the appended claims.

### BRIEF DESCRIPTION OF THE DRAWINGS

FIG. 1 illustrates a serum bottle and a magnetic Teflon spinner to mushroom cultivation according to a preferred embodiment of the present invention.

FIG. 2 illustrates a polycarbonate (PC) bioreactor and a rotating shaker according to the above preferred embodiment of the present invention.

### DETAILED DESCRIPTION OF THE PREFERRED EMBODIMENT

The present invention provides a method of producing mushroom beta-glucan which comprises the following steps.

(a) Grow mycelium from mushroom strains.

(b) Make a culturing liquid from the mycelium.

(c) Dispose the culturing liquid in one or more bottles 10 for a first time fermentation under a first predetermined condition.

(d) After the culturing liquid is fermented, mix and stir the culturing liquid with a new culturing medium in each of the bottles, wherein said culturing medium is driven to spin at a predetermined spinning speed to control an amount of oxygen being dissolved in the culturing liquid and to create shear force to make the mycelium to densely grow and reproduce at a higher growth rate.

(e) Second time ferment the culturing liquid after the mycelium and beta-glucan reach highest concentration levels thereof, wherein said mycelium is shredded to reduce a size thereof after having fermented twice.

(f) Implant said mycelium into one or more bioreactors 30 each of which contains an extract liquid culturing medium.

(g) Ferment the mycelium in each of the bioreactors under a predetermined condition, wherein the fermentation is stopped when a stationary phase is reached to obtain the highest concentration of mycelium cell and beta-glucan.

In the step (a), preferably, the collected mushroom strains are placed on the slanted side of the solid medium and transferred to YM agar medium under an aseptic condition. The composition of the YM agar medium includes 0.3% (w/w) yeast extract, 0.3% malt extract, 0.5% peptone, 1.0% Dextrose and 1.5% Agar. The mushroom strains on YM agar medium are incubated in a 28° C. incubator, and mycelium starts to appear in 2 to 3 days and fills the culture medium in about a week.

In an aseptic bench, a small piece of solid mycelium medium stated above is put into approximately 20 ml of new, cold and sterilized culturing solution in 80 T of the T-shaped animal cell culturing serum flask which is slightly shaken and has a lid slightly opened for air exchange. The flask is then placed in a 28° C. incubator for a week and better and faster growth strains are selected for liquid culturing species in the step (b).

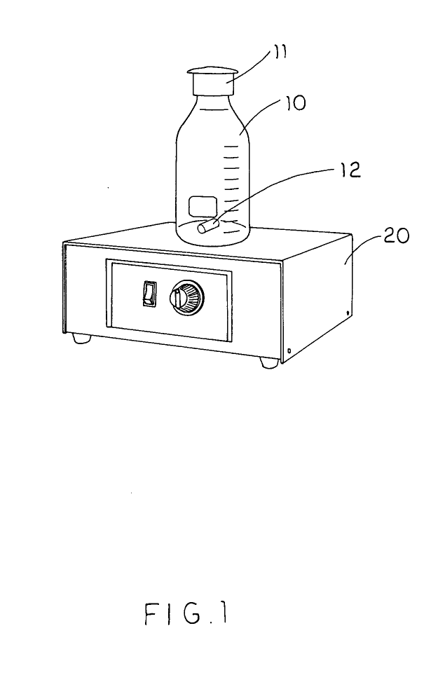

According to a preferable embodiment of the present invention, in the step (c), as shown in FIG. 1, the bottle 10 is embodied as a 1 L serum bottle with a one-direction ventilating silicate stop 11 and a magnetic stirring unit 12 such as a Teflon spinner with 1 centimeter in diameter and 4 centimeter long is placed in the serum bottle 10. Thereafter, 800 ml of YM culturing liquid is poured into the serum bottle 10 under a high temperature at 121° C. and high pressure aseptic condition for 15 minutes, and waiting for the bottle 10 cooling down.

After fermentation, in the step (d), the liquid culture is poured out into a new culturing medium and inoculated to the previously stated serum bottle 10. Under room temperature, the serum bottle 10 is placed on top of a magnetic stirrer 20 which makes the magnetic stirring unit 12 to spin at the speed of approximately 300 rpm, so as to evenly mix the content therein. Also, the amount of dissolved oxygen is determined by the spinning speed of the magnetic Teflon spinner 12. The shear force created by the spinner 12 makes the mycelium to densely grow and reproduce at a higher rate. When the mycelium and beta-glucan reach their highest concentration level, the fermentation process should be terminated and the liquid culture is transferred to another 10 L serum bottle 10 containing 8 L culturing medium for another fermentation scale-up process.

Having fermented twice, the mycelium is shredded by the magnetic spinner 12 to reduce the size of the mycelium in the step (e), such that the size thereof is not too large to affect future fermentation process and at the point, the selected species are ready to be implanted into a prepared polycarbonate (PC) bioreactor 30.

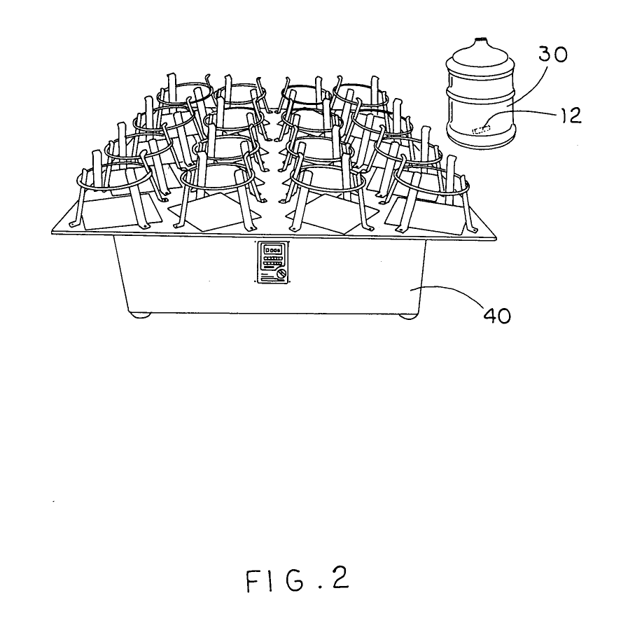

In the step (f), the bioreactor 30 contains 4% glucose and 0.5% yeast extract liquid culturing medium, and is secured at a rotating (orbital) shaker 40 for rotating the liquid culturing medium. In the step (g), as shown in FIG. 2, adjust a rotating speed to approximately 80 rpm and allow sufficient source of dissolved oxygen and stirring. Under room temperature, leave fermentation to take place until its growth curve reaches the stationary phase that the number of mycelium cells and beta-glucan concentration is their highest.

According to the present invention, the producing method is capable of producing beta-glucan and other metabolites from all types of mushrooms via liquid culture, wherein a uniform liquid culture medium, along with the usage of serum bottles 10 with magnetic stirring bars 12 and non-toxic polycarbonate (PC) bioreactor 30 is arranged to allow the mushrooms growing well and yielding high beta-glucan production rate, and thus reduce setup cost for cultivating liquid beta-glucan from mushrooms.

According to the present invention, the serum bottle 10 with a one-direction ventilating silicate stop and the magnetic Teflon spinner 12 are used such that shear force created by the spinner makes the mycelium to densely grow and reproduce at a higher rate.

As of today, most submerged liquid cultures for mushroom beta-glucan and other metabolites are produced in a sequence of bioreactors after initial mycelium production. Such production scheme involves massive investment in facility and the occurrence of the contamination during the process may incur great loss.

In mushroom liquid fermentation, carbon, nitrogen source and growth factor in the culturing medium are the key factors to the rate of beta-glucan production. Most production methods use cheaper materials or add some special compounds to increase the production rate, but this would result in more purification steps and creates additional waste. Therefore, the present invention adopts food-grade materials and follows simple recipe so that the culturing medium contains nothing more than glucose and yeast extracts. Experiments have shown that the present invention is suitable for all types of mushroom growth and the production rate of beta-glucan is good. After fermentation, the mushrooms only need minimum purification procedure for direct consumption, and with further processing, the mushrooms can become different products for medical, healthy or cosmetic use.

Agar plates are commonly used in subculturing methods. During fermentation, it is important to speed up the production of usable inoculums to further scale-up inoculation. The present invention provides a method of mushroom cultivation which uses the 80 T, the T-shaped tissue culture vessel for animal cell culture. The present invention has a faster and higher duplicating or reproducing rate, and if there is contamination, it can be detected at the early stage. Different from other liquid fermentation methods which shake the flasks, the present invention uses serum bottle 10 with the air-permeable silica stop 11 and the Teflon magnetic spinner 12 for mixing. The present invention prompts extra-cellular beta-glucan production, reduces the formation of mycelium block and thus increases the amount of dissolved oxygen and material transportation rate.

Most conventional submerging culture methods use a stainless steel bioreactor with connection tubing to an industrial size boiler, and all of which contribute to a significant cost. In the present invention, culture medium is contained in a transparent and thermal resistive polycarbonate (PC) bioreactor 30 and covered with the air-permeable silica stop 11 and thus the bioreactor 30 can be sterilized in a miniature autoclave. Such a bioreactor holds up to 16 L of fermentation volume, which is larger than conventional tabletop bioreactor. Furthermore, the PC bioreactors 30 are lighter and cheaper than conventional bioreactors.

After cooling down from the sterilization process, mushroom stains from the serum bottle 10 are inoculated to the PC bioreactor placed on a custom-made rotating shaker. The rotating shaker 40 can hold up to 16 bioreactors 30 to increase the yields and more than one kinds of mycelium can be cultivated at the same time. The method of the present invention is extremely flexible and can be used for either research or manufacturing purpose.

Polycarbonate PC is the preferably material for the bioreactor 30 while PC material is light, non-toxic, thermal resistant, transparent, and durable. Thus, PC reactors are excellent substitutions of the expensive stainless bioreactors. During fermentation, the PC bioreactor 30 enables the user to monitor the entire fermentation process such that the production process can be better controlled and screened. The shaking rate, amount of dissolved oxygen and growth conditions can all be adjusted accordingly. Experimental productions with mushroom-like Ganoderma lucidum, Coriolus Versicolor and Lentinula edodes have good yields and beta-glucan production is also shown to have good production rate.

The present invention is suitable for the production of beta-glucan and other metabolites from all types of mushroom -via the liquid culture method; The spinning-speed of the magnetic spinner 12 and the liquid volume can be adjusted to control the property of dissolved oxygen. The producing method of the present invention is applicable to a wide range of cultivation, such as obligated aerobic, aerobic and anaerobic microbial fermentation, and for both animal and flora cell cultivation. The PC bioreactor also allows liquid cultivation of algae and Rhodobacter Capsulatus under special light sources.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of producing mushroom beta-glucan, comprising the steps of:
(a) growing mycelium from mushroom strains;
(b) making a culturing liquid from said mycelium;
(c) disposing said culturing liquid in one or more bottles for first time fermentation under a first predetermined condition;
(d) after said culturing liquid is fermented, mixing and stirring said culturing liquid with a culturing medium in each of said bottles at a predetermined spinning speed to control an amount of oxygen being dissolved in said culturing liquid and to create shear force to make said mycelium to densely grow and reproduce;
(e) after said mycelium and beta-glucan reach predetermined concentration levels thereof, second time fermenting said culturing liquid and then shredding said mycelium to reduce a size thereof;
(f) implanting said mycelium into one or more bioreactors, each of which is made of non-toxic, thermal resistant, transparent, durable material and contains a liquid culturing medium; and
(g) fermenting said mycelium in each of said bioreactors while allowing sufficient source of dissolved oxygen and stirring until a stationary phase to obtain a predetermined concentration of mycelium cell and beta-glucan.

2. The method as recited in claim 1 wherein, in the step (f), each of said bioreactors is a polycarbonate (PC) bioreactor.

3. The method, as recited in claim 2, wherein said liquid culturing medium of each of said polycarbonate (PC) bioreactors contains 4% glucose and 0.5% yeast extract liquid culturing medium.

4. The method as recited in claim 1 wherein, in the step (g), said mycelium is stirred at a spinning speed-about 80 rpm under-room temperature until said fermentation reaches said stationary phase.

5. The method as recited in claim 3 wherein, in the step (g), said mycelium is stirred at a spinning speed about 80 rpm under room temperature until said fermentation reaches said stationary phase.

6. The method, as recited in claim 1, wherein the step (b) comprises the steps of:
(b.1) putting said mycelium is into a cold and sterilized culturing solution in a T-shaped animal cell culturing serum flask;
(b.2) slightly shaking said flask, wherein a lid of said flask is slightly opened for air exchange; and
(b.3) placing said flask in a 28° C. incubator for a week, wherein better and faster growth strains are selected for liquid culturing species.

7. The method, as recited in claim 3, wherein the step (b) comprises the steps of:
(b.1) putting said mycelium is into a new, cold and sterilized culturing solution in a T-shaped animal cell culturing serum flask;
(b.2) slightly shaking said flask, wherein a lid of said flask is slightly opened for air exchange; and
(b.3) placing said flask in a 28° C. incubator for a week, wherein better and faster growth strains are selected for liquid culturing species.

8. The method, as recited in claim 5, wherein the step (b) comprises the steps of:
(b.1) putting said mycelium is into a cold and sterilized culturing solution in a T-shaped animal cell culturing serum flask;
(b.2) slightly shaking said flask, wherein a lid of said flask is slightly opened for air exchange; and
(b.3) placing said flask in a 28° C. incubator for a week, wherein better and faster growth strains are selected for liquid culturing species.

9. The method as recited in claim 1 wherein, in the step (c), said culturing liquid is transferred into each of said bottles with a one-direction ventilating silicate stop, wherein said culturing liquid is contained in each of said bottles for 15 minutes at 121° C. is then waited for each of said bottles being cooled down.

10. The method as recited in claim 5 wherein, in the step (c), said culturing liquid is transferred into each of said bottles with a one-direction ventilating silicate stop, wherein said culturing liquid is contained in each of said bottles for 15 minutes at 121° C. is then waited for each of said bottles being cooled down.

11. The method as recited in claim 8 wherein, in the step (c), said culturing liquid is transferred into each of said bottles with a one-direction ventilating silicate stop, wherein said culturing liquid is contained in each of said bottles for 15 minutes at 121° C. is then waited for each of said bottles being cooled down.

12. The method as recited in claim 1 wherein, in the step (a), said mushroom strains are placed in YM agar medium which contains 0.3% (w/w) yeast extract, 0.3% malt extract, 0.5% peptone, 1.0% Dextrose and 1.5% Agar, wherein said mushroom strains on YM agar medium are incubated in a 28° C. incubator to grow said mycelium as a uniform liquid culture medium.

13. The method as recited in claim 5 wherein, in the step (a), said mushroom strains are placed in YM agar medium which contains 0.3% (w/w) yeast extract, 0.3% malt extract, 0.5% peptone, 1.0% Dextrose and 1.5% Agar, wherein said mushroom strains on YM agar medium are incubated in a 28° C. incubator to grow said mycelium as a uniform liquid culture medium.

14. The method as recited in claim 11 wherein, in the step (a), said mushroom strains are placed in YM agar medium which contains 0.3% (w/w) yeast extract, 0.3% malt extract, 0.5% peptone, 1.0% Dextrose and 1.5% Agar, wherein said mushroom strains on YM agar medium are incubated in a 28° C. incubator to grow said mycelium as a uniform liquid culture medium.

15. The method as recited in claim 1 wherein, in the step (d), each of said bottles is placed on top of a magnetic stirrer and contains a magnetic stirring unit which is driven by said magnetic stirrer to spin so for mixing and stirring said culturing liquid.

16. The method, as recited in claim 15, wherein said magnetic stirring unit is a magnetic Teflon spinner.

17. The method as recited in claim 11 wherein, in the step (d), each of said bottles is placed on top of a magnetic stirrer and contains a magnetic stirring unit which is driven by said magnetic stirrer to spin so for mixing and stirring said culturing liquid.

18. The method, as recited in claim 17, wherein said magnetic stirring unit is a magnetic Teflon spinner.

19. The method as recited in claim 14 wherein, in the step (d), each of said bottles is placed on top of a magnetic stirrer and contains a magnetic stirring unit which is driven by said magnetic stirrer to spin so for mixing and stirring said culturing liquid.

20. The method, as recited in claim 19, wherein said magnetic stirring unit is a magnetic Teflon spinner.

21. The method as recited in claim 1 wherein, in the step (d), said culturing liquid is spun at a spinning rate of about 300 rpm to evenly mix said culturing liquid, to determine said amount of oxygen being dissolved in said culturing liquid, and to create said shear force to densely grow and reproduce said mycelium.

22. The method as recited in claim 15 wherein, in the step (d), said culturing liquid is spun at a spinning rate of about 300 rpm to evenly mix said culturing liquid with said culturing medium, to determine said amount of oxygen being dissolved in said culturing liquid, and to create said shear force to densely grow and reproduce said mycelium.

23. The method as recited in claim 17 wherein, in the step (d), said culturing liquid is spun at a spinning rate of about 300 rpm to evenly mix said culturing liquid with said culturing medium, to determine said amount of oxygen being dissolved in said culturing liquid, and to create said shear force to densely grow and reproduce said mycelium.

24. The method as recited in claim 19 wherein, in the step (d), said culturing liquid is spun at a spinning rate of about 300 rpm to evenly mix said culturing liquid with said culturing medium, to determine said amount of oxygen being dissolved in said culturing liquid, and to create said shear force to densely grow and reproduce said mycelium.

25. The method as recited in claim 1 wherein, in the step (f), said bioreactors are secured at an orbital shaker.

26. The method as recited in claim 4 wherein, in the step (f), said bioreactors are secured at an orbital shaker.

27. The method as recited in claim 5 wherein, in the step (f), said bioreactors are secured at an orbital shaker.
